(19) United States
(12) Patent Application Publication
(10) Pub. No.: US 2023/0404615 A1
(43) Pub. Date: Dec. 21, 2023

(54) PROBE-GUIDE FOR IDENTIFYING AND HARVESTING A BIOPSY

(71) Applicant: FLUOLINK AB, Linköping (SE)

(72) Inventors: Karin WÅRDELL, Linköping (SE); Johan RICHTER, Linköping (SE); Håkan ROHMAN, Mantorp (SE); Hauke VAN DE KOOIJ, Onsala (SE)

(21) Appl. No.: 18/246,705

(22) PCT Filed: Oct. 1, 2021
(86) PCT No.: PCT/SE2021/050971

§ 371 Date: Mar. 27, 2023

(30) Foreign Application Priority Data
Oct. 1, 2020 (SE) 2051151-5

(51) Int. Cl. A61B 17/34; A61B 10/02; A61B 34/20

(52) U.S. Cl. CPC A61B 17/3403; A61B 2034/2051; A61B 34/20; A61B 10/0275

(57) ABSTRACT

The present invention provides a probe-guide for guiding, identifying, and harvesting a biopsy, said probe-guide can be combined with an optical probe, a biopsy needle or a device for delivering a medicament.

### TECHNICAL FIELD

The present invention relates to a device, a probe-guide, for localizing, identifying, and harvesting a biopsy, a method for harvesting a biopsy and a system thereof. More particularly, the technical field relates to neurosurgery.

### BACKGROUND OF THE INVENTION

Proper diagnosis of most cancers requires closer examination and a biopsy is harvested to collect cells for examination. Brain tumor biopsies are usually performed by using frameless navigation systems or stereotactic frames.

WO 2017/135873 A1 discloses a forward-looking probe suitable for use in a stereotactic frame, the probe is used for identifying the best site for harvesting a biopsy in a tumor, thereby reducing the risk for obtaining a biopsy not reflecting the malign tissue. The probe use fluorescence for identifying malign tissue (PpIX), and blood vessels by laser Doppler flowmetry, thereby also reducing the risk for hemorrhage. The probe provides a pre-made trajectory to the tumor site and is removed from the trajectory before the biopsy needle is inserted into the trajectory to harvest the biopsy. The drawback with this device is that at least two insertions into the tissue are required before the biopsy is harvested.

US2015148629 A1 discloses a system for stereotactic biopsies, an optical spectroscopy probe is placed inside a biopsy device having an outer cannula and an inner cannula with cutting edges. One embodiment comprises a removably probe, and the mechanical biopsy is performed from the side wall of the guide. The problem with this embodiment is for example that the side-looking guide miss blood vessels in front of it which can cause hemorrhage.

Drawbacks with the prior art is that several insertions into the tissue are required before a biopsy reflecting the malignity of a tumor is harvested, side-looking guides miss blood vessels in front of them which can cause hemorrhage.

Each step of insertion and withdrawal operations of instruments in an open skull increase the risk for complications, such as for example infection or hemorrhage. Moreover, more specialists are required to be consulted from the operation room which is time consuming and thus more costly for the society.

Hence, safer and more effective devices performing all necessary steps for harvesting a biopsy with only one insertion into the brain are needed.

### SUMMARY OF THE INVENTION

The present invention provides a device, i.e., a probe-guide for guiding, identifying, and harvesting a biopsy from a tumor. The probe-guide is used for providing a trajectory into a brain tumor, for example for harvesting a biopsy. The present probe-guide is designed to host a second device, such as for example an optical probe, a biopsy needle and a device for administering a drug. By using the present probe-guide in combination with the removably devices above, only one insertion into the brain is needed.

The present invention provides a probe-guide for guiding, identifying, and harvesting a biopsy, the probe-guide is substantially tube-shaped, having a distal and a proximal end wherein the distal end is for insertion into a body and the proximal end is for connection to an energy source.

The wall of the probe-guide comprises a first and a second opening, the first opening is arranged in the distal end or tip, pointing in an insert direction (forward-looking). The second opening is arranged at a distance from the distal end or tip, preferably arranged in the (elongated) sidewall of the probe-guide, thus pointing sideways (side-looking).

The probe-guide is designed to receive another device into its lumen such as:
i) a removable optical probe (probe) for optical guidance and/or identification of malign tissue, and/or
ii) a removable biopsy needle for harvesting a biopsy,
The probe is essentially tube-shaped outer housing accommodating optical fibers, having a distal and a proximal end, wherein the distal end is for insertion into a body and the proximal end is for connection to an energy source. Optical fiber pairs are arranged in close proximity towards the distal end. The optical fibers at least 4. The diameter of the fibers are in the range of 125 to 250 μm. The probe is removably arranged into the lumen of the probe-guide and configured to transmit and receive light via the first opening of the probe-guide.

The biopsy needle is removably arranged into the lumen of the probe-guide and configured to harvest a biopsy via the second opening, wherein the probe and the biopsy needle are not arranged in the lumen of the probe-guide simultaneously.

In a second aspect the length of the probe-guide is in the range of 100-300 mm.

In a third aspect, the width (diameter) is in the range of 1.5-3 mm.

In yet another aspect the probe (i) have the length in the range of 50-400 mm.

The probe comprises a light spectrometer laser; and
a LDF laser; and
a Raman laser; and
a spectrometer detector; and
a laser doppler detector; and
a Raman detector.

The typical diameter (Ø) of the fibers is in the range of 100-250 μm, 125-200 μm.

In one aspect, the probe-guide and probe described above, are preferably made of medical steel.

In one embodiment, the comprises the probe-guide a pointer for electromagnetic navigation. The pointer is arranged into the lumen of the probe-guide.

In yet another embodiment is a device for delivering a medicament arranged into the lumen of the probe-guide.

Another object is to provide a method for guiding, identifying, and harvesting a biopsy comprising the following steps:
i) mounting a stereotactic device or frameless navigation system;
ii) drilling a hole at a predetermined site in a skull (predicted from the MR-images);
iii) moving the probe-guide comprising a removable optical probe described above to the pre-drilled hole via a navigation system or by an electrical or hand driven mechanical device, or by hand, or by a robot navigation system;
iv) deploying the probe-guide comprising the probe into the pre-drilled hole with or without a supporting device;
v) measuring and recording the microvascular perfusion, TLI and fluorescence stepwise with or without an electrical or mechanical insertion device or continuously along the way to the target;
vi) pushing the probe probe-guide forward and record if the microcirculation is within a valid level meaning that no vessels are detected in front of the probe-guide;
vii) determining where the biopsy is to be taken by measuring and recording emitted fluorescence light (every mm) before and throughout the tumour trajectory wherein the emitted fluorescence signal is illustrated as a peak or as an audio signal, where the high fluorescence signal indicates highly malignant tissue of said tumour, and
viii) removing the probe from the probe-guide,
ix) arranging the biopsy-needle into the lumen of the probe-guide,
x) harvest the biopsy via the opening (f) in the sidewall of the probe-guide,
xi) removing the probe-guide comprising the biopsy needle
xii) sewing the wound together, and
xiii) sending the biopsy for further examination.

In one embodiment of the method for guiding, identifying, and harvesting a biopsy comprising steps i)-ix) above further comprising the steps of:
removing the biopsy needle in step xi);
arranging a device comprising a medicament in the lumen of the probe-guide;
delivering the medicament to the target site, and
sewing the wound together.

A third object of the present invention is to provide a real time system for localizing, identifying, and harvesting a tumor biopsy, said system comprising:
i) a probe-guide, a removably probe, a removably biopsy-needle as described above,
ii) a stereotactic or frameless system,
iii) a control device, and
iv) an analysis system.

In yet another embodiment comprises the probe-guide described above openings for transmitting signals from a device arranged or inserted into the lumen of the probe-guide, for removal of tissue by vaporization, abrasion, or destruction.

### BRIEF DESCRIPTION OF THE DRAWINGS

FIG. 1 Shows a schematic drawing on the probe-guide comprising a probe.

FIG. 2 Shows a cross-section A-A of the probe-guide comprising a probe in FIG. 1 and the fiber configuration inside the probe.

### DETAILED DESCRIPTION OF THE INVENTION

In the following, the present invention will be described in more detail by way of embodiments and with reference to the accompanying drawings.

Other features and uses of the invention and their associated advantages will be evident to a person skilled in the art upon reading the description and the examples.

It is to be understood that the present invention is not limited to the embodiments shown here. The following examples are provided for illustrative purposes and are not intended to limit the scope of the invention, since the scope of the present invention is limited only by the appended claims and equivalents thereof.

If nothing else is defined, any terms and scientific terminology used herein are intended to have the meanings commonly understood by those of skill in the art to which this invention pertains.

A biopsy is a procedure in which a piece of tissue or a sample of cells is removes from your body, the sample will thereafter be analysed in a laboratory.

Frameless navigation is used for both optical and electromagnetic navigation.

Stereotactic frames cover centre of arc and different frames.

The term probe or optical probe is in this application defined to be a probe comprising fibers for spectrometer laser and spectrometer detector, laser doppler flowmetry and laser doppler flowmetry detector, and Raman laser and Raman detector, and fluorescence measurements.

A fluorophore (or fluorochrome, similarly to a chromophore) is a fluorescent chemical compound that can re-emit light upon light excitation, for example 5-ALA and Sodium (LF).

The term probe-guide 1 is in this application meant to be a device that guides and prepares a trajectory in for example brain tissue for identifying and/or harvesting a biopsy or deliver a medicament. The probe-guide 1 comprises openings for transmitting signals from a device arranged or inserted into the probe-guide. The probe-guide 1 can also be used for removal of tissue by vaporization, abrasion, or destruction.

Methods used include heating tissue by hot liquids or microwave thermal heating, freezing (cryoablation), chemical ablation, and photoablation with laser. The probe-guide 1 may also be used for freehand biopsy at e.g. open surgery. The surgery may be performed under microscope (e.g. FL400 or FL560) for amplifying the signals.

Moreover, the probe-guide may comprise a probe for electro-physiological measurements and stimuli during surgery.

A biopsy needle 3 is an instrument for harvesting a biopsy, for example a needle, a punch, or a pincette. The biopsy needle(s) discussed here are commercial and suitable to be inserted into the probe-guide without further modification(s). The distance from the tip to the start of the side-opening may be in the range of 2-4 mm, preferably about 2.5 mm. The side-opening (to harvest the tissue) is about 6-10 mm long, preferably 9 mm long.

For example, the distance from the tip to the middle of the side-opening of the biopsy needle is 7 mm (Medtronic), this biopsy needle is commonly used in a Stealth8-navigation system.

A device for delivering a medicament 4 means any device suitable to be inserted into the probe-guide 1, e.g., a tube-shaped device having a distal and a proximal end, wherein the distal end fit the distal end/tip of the probe-guide 1. The medicament can be released from the device via an opening corresponding to opening e or f of the probe-guide 1.

### Example 1

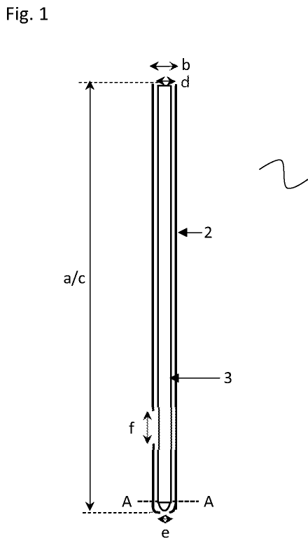

FIG. 1 shows one embodiment of the probe-guide 1 of the present application. The probe-guide 1 comprises in this embodiment an optical probe 2. The probe 2 is removably arranged into the lumen of the probe-guide 1 and can be exchanged to for example a biopsy needle 3, a device for delivering a medicament 4 or a device for electrophysiological measurements etc.

The probe-guide 1 is essentially tube-shaped, having a distal and proximal end. The distal end is for insertion into a body and the proximal end is for connection to an energy source.

The probe-guide 1 is substantially hollow and designed to receive another device, for example an optical probe 2, a biopsy needle 3 and a device for delivering a medicament 4. The optical probe 2 makes it possible to monitor a trajectory through the first opening e during the insertion. Vessels in front of the probe-guide will also be detected by the probe-guide 1 comprising the probe 2.

In one embodiment is a fluorophore used to detect/identify the tumor cells, for sodium fluorescein (FL).

In one embodiment is a photosensitive drug used to detect/identify the tumor cells, for example 5-aminolevulinic acid (5-ALA) which is converted to Protoporphyrin IX a fluorescent substance that accumulates in tumor cells and can easily be visualized.

The biopsy needle 3 will harvest a biopsy from the tissue via the second opening f, this will occur when the probe-guide/probe 3 is in the target position.

The length a of the probe-guide 1 is in this embodiment 100-300 mm, but other lengths are also possible. The width (diameter) b is in the range of 1.5-3 mm. This length and width make the probe-guide 1 suitable to fit both frameless navigation systems and stereotactic systems as well.

The distal end or tip of the probe-guide 1 is for example of U-shape.

The wall of the probe-guide 1 comprises a first e and a second f opening. The first opening e is arranged in the distal end or tip (see FIG. 1), pointing in an insert direction (forward-looking). The second opening f is arranged at a distance from the distal end or tip, preferably arranged in the longitudinal sidewall of the tube-shaped probe-guide 1, thus pointing sideways (side-looking).

The second opening f is arranged in the sidewall about 2-4 mm from the distal end/tip.

The opening e should have a size that enables the optical probe comprising fibres looking through the opening, the opening e may be in the range of 0.5-2.5 mm.

The opening f may be in the range of 5-10 mm. The diameter or length is typically 7 or 8 mm for standard biopsy kits. The shape of the openings e, f may be of any suitable shape.

The first opening e pass the light(s) from for example an optical probe to the tissue. The arrangement of the opening e into the distal end or tip makes the probe-guide 1 forward-looking, and can thus identify for example blood vessels in front of the probe-guide 1 when it is inserted into the tissue and moves along a trajectory towards the biopsy site. If fluorophores are used, these can also be identified closer to the target, i.e., the malign tissue.

The probe-guide 1 is designed to receive for example an optical probe 2 comprising fibers reaching to the first opening e for monitoring a trajectory through the first opening e during the insertion.

A probe 2 having an essentially tube-shaped outer housing accommodating optical fibers can be removably inserted into the probe-guide 1. The probe 2 has a distal and a proximal end. The distal end is for insertion into a body and the proximal end is for connection to an energy source, i.e., the distal end of the probe 2 coincides with the distal end of the probe-guide 1, and the fibers coincides with the first opening e so it can look forward through the opening e, when properly arranged into the probe-guide 1.

The length (c) of the probe 2 is in the range of 50-400 mm, or 100-300 mm i.e., the same length or longer than the length of the probe-guide (a). However, it may also be shorter than the probe-guide.

The width (d) of the probe 2 should fit to be arranged into the inner part of the probe-guide 1 (b).

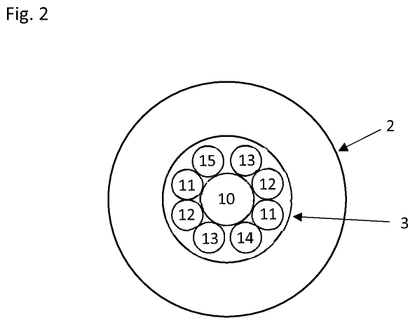

FIG. 2 shows a cross-section of the dashed line A-A close to the distal end/tip of the probe-guide 1 comprising the optical probe 2 in FIG. 1. The optical fiber pairs are arranged in close proximity towards the distal end. In one embodiment the optical fibers are at least 4. In yet another embodiment at least 6 optical fibers, or 8 optical fibers. The diameter of the fibers are in the range of 50 to 250 μm, for example 200 μm, and 125 μm.

Up to 9 fibres are possible to arrange in the probe 2. The fibers can be connectable to LDF, Fluorescence, DRS and Raman and Raman spectroscopy.

One fiber of the fiber pair is operatively connected via the proximal end to a light source.

The other fiber of the fiber pair is operatively connected via the proximal end to a detector.

The light source may be a spectrometer laser 10. The fiber 10 has for example a diameter (Ø) of 200 μm.

The light source may be a blue laser (405 nm) for the fluorescence.

The light source may be a laser in the red or near infrared region (780 nm).

The light source may be a LDF laser 12.

The light source may be a Raman laser 14.

The detector may be a spectrometer detector 11.

The detector may be a laser doppler detector 13.

The detector may be a Raman detector 15.

The fibers 11-15 may have a diameter (Ø) in the range of 100-250 μm, in one example is the diameter (Ø) about 125 μm.

The fiber 10 may have a diameter (Ø) in the range of 100-250 μm, preferably about 200 μm.

The material of the probe-guide 1 and probe 2 should be medical steel, but an alternative solution could be printed with 3D using medical plastic material and other maintenance methods.

The probe-guide 1 is also designed to receive a biopsy needle 3. The second opening f is arranged for allowing the biopsy needle 3 to harvest a piece of tissue when the probe-guide 1 comprising the biopsy needle 3 is in a target position.

The needle of the biopsy needle 3 should have a sharp edge fitting the opening fin the side wall of the probe-guide 1. The biopsy needle 3 can be any suitable commercial biopsy needle.

A pointer for electromagnetic navigation may be arranged inside the probe-guide 1.

In yet another embodiment, a device for delivering a medicament can be arranged into the probe-guide 1. Brain tumors situated relatively deep in the brain may be treated by delivering an anti-cancer drug directly to the tumor site, after delivery of the drug, the wound is stitched together. The device could for example be designed as a tube-shaped syringe with a piston for releasing the medication at the target site.

In another embodiment the probe-guide 1 comprising the optical probe 2 can be used in therapy, for example Photodynamic therapy (PDT), a form of phototherapy involving light and a photosensitizing chemical substance, used in conjunction with molecular oxygen to elicit cell death (phototoxicity).

### Example 2

Use of the probe-guide 1 for guiding, identifying, and harvesting a biopsy from a brain tumor in a safe way and with only one insertion into the brain.

The probe-guide 1 comprising the probe 2 (as described above) is inserted into an opening in the skull and provides a trajectory through the brain, the probe 2 identifies blood vessels in front of the probe during the insertion to the site, and fluorophores if used also malign tissue. When the probe-guide 1 comprising the probe 2 reach the target site, the probe 2 is removed from the probe-guide 1, and if the aim is to harvest a biopsy, a biopsy needle 3 is arranged into the lumen of the probe-guide 1, without moving the probe-guide 1 from the site. The biopsy needle 3 harvest the biopsy through the second opening f. The biopsy needle 3, and the probe-guide 1 or the probe-guide 1 comprising the biopsy needle 3 is removed, the wound is stitched together, the biopsy is sent for detailed examination. The superior advantage with the present guide-probe (1) and removably devices (2, 3, 4 etc), method and system is that the malignity of the tissue is confirmed during the surgery. This means that the surgeon/staff does/do not have to wait for a preliminary examination of the harvested biopsy before stitching the wound together. Thereby minimizing the risk to have to make yet another trajectory and to harvest yet another biopsy i.e., the risk of repeating the entire procedure.

In another embodiment, before the probe-guide 1 is withdrawn from the trajectory the probe 2 or the biopsy-needle is removed and exchanged to a device 4 comprising a medicament, for example an anti-cancer drug. The medicament is delivered to the site via one of the openings e, f before the guide 1 comprising the device 4 is removed and the wound is stitched together.

The present invention further provides a real time system for localizing, identifying, and harvesting a biopsy at the best site, said system comprising:
the probe-guide 1 comprising means for measuring and recording the microvascular perfusion in the normal brain structure and in the tumour; and
i) a control device.

The system may further comprise an analysis system.

Finally, the present invention further provides a method for guiding, identifying, and harvesting a biopsy from a subject. Said method comprises the steps of:
ii) administering for example 5-ALA in a dose of 5-20 mg/kg to a subject prior to surgery,
iii) mounting a stereotactic device or a frameless system,
iv) drilling a hole at a predetermined site in a skull (predicted from the MR-images),
moving the probe-guide 1 comprising the probe 2 to the pre-drilled hole via a navigation system,
deploying the probe-guide 1 comprising the probe 2 into the pre-drilled hole with or without a supporting device,
v) measuring and recording the microvascular perfusion (LDF), and fluorescence via opening e stepwise or continuously along the way to the target,
vi) pushing the probe-guide 1 forward and record if the microcirculation is within a valid level meaning that no vessels are detected in front of the probe,
vii) determining where the biopsy is to be taken by measuring and recording emitted fluorescence light (every mm) before and throughout the tumour trajectory wherein the emitted fluorescence signal is illustrated as a peak or as an audio signal, where the high fluorescence signal indicates highly malignant tissue of said tumour, and
   removing the probe 2 from the probe-guide 1;
   arranging a biopsy needle 3 into the probe-guide 1;
   harvesting a biopsy via opening f,
   removing the probe-guide 1 comprising the biopsy needle 3 from the trajectory,
   sew the wound together.

In another embodiment, the biopsy needle 3 is removed from the probe-guide 1, and the device 4 comprising a medicament is arranged in the probe-guide 1, after delivery of the medicament, the probe-guide 1 comprising the device 4 is removed, and the wound is stitched together.

This means that the disclosed probe-guide 1 is designed to receive for example an optical probe 2 as defined above into its lumen, thus blood vessels can be detected and avoided in front of the forward-looking probe 1 during insertion to the site of interest, and malign tissue can be identified if fluorophores are used (see above). The probe-guide 1 can also be side-looking via an opening in the side of the probe-guide 1. When the probe-guide 1 comprising the probe 2 reached the target site, the probe 2 is removed from the probe-guide 1. The probe-guide 1, still arranged at the target site, can now receive for example a biopsy needle 3 into its lumen, and a biopsy can be harvested through the second opening f of the probe 1. The biopsy needle 3, or the probe-guide 1 comprising the biopsy needle 3 can thereafter be removed. The probe-guide 1 is designed to smoothly enter brain tissue and can be adapted to different tasks by insertion of different functional units or devices such as an optical probe 2, a biopsy needle 3, a housing or container comprising a medicament 4 etc.

### Example 3

### Stereotactic Biopsy Procedure for Identifying the Best Site for Harvesting a Biopsy

Prior to surgery the patient is given a 5-20 mg/kg dose of 5-aminolevulinic acid (5-ALA), and the stereotactic frame is attached to the patient's head before MRI. 5-ALA accumulates as protoporphyrin IX (PpIX) in tumour cells.

Target and the trajectories towards the tumor are calculated by use of surgical planning software (e.g., Surgiplan (Elekta AB), Stealth system (Medtronics) and transformed to the co-ordinates of the stereotactic system. After skull opening, the probe-guide1/probe 2 is inserted in 1 mm steps with a mechanical device by the surgeon, (manual insertion and insertion with a robot are alternatives), from cortex towards the tumor core. Each step is followed by a recording of the microcirculation and total light intensity (TLI) with the LD. When approaching the suspected lesion, the fluorescence of PpIX is measured and in case of tumor tissue a fluorescence peak is immediately visible in the OR. The peak at 635 nm is an indicator of malignancy. The data collection is done by the surgeon or other medical staff. When the entire trajectory has been passed, the optical probe 2 is retracted and replaced by the biopsy needle 3 which is carefully inserted towards the suspected lesion. Biopsies are then taken according to the clinics' routine, i.e. at 3-5 positions along the trajectory. By using the mechanical device, the exact position for the optical recordings and biopsies can be defined and matched also to the preoperative MRI in the postoperative analysis of the collected data. It is also possible to stop the insertion if the blood flow indicates a vessel structure i.e. high blood flow in front of the probe tip (FIG. 2b).

In another embodiment the patient is given sodium fluorescein (FL). FL is safely used in fluorescence-guided microsurgery for imaging various brain tumors. Under the YELLOW 560 nm surgical microscope filter, low-dose FL as a fluorescent dye helps in visualization. FL accumulates in tumour areas with disrupted blood brain barrier that can then be visualized under a microscope with dedicated filters.

The healthcare cost is reduced due to reduced time needed for surgery and health care professionals, decreased risk for misdiagnosis, reduced risk for the need of repeating the procedure for harvesting a biopsy for diagnosis, before removing the tumour by surgery.

In summary, the present invention provides a device 1, that makes it possible to safely guide, identify and obtain a biopsy with only one insertion into the body, e.g., the brain.

Tumor tissue and vessel structures are identified which decrease surgical time and at the same time increase efficacy and safety.

The number of trajectories is reduced by using said device 1.

The probe-guide 1 is compatible with common systems used today, such as frameless navigation and stereotactic systems.

The tissue can be identified in a safe and reliable way with frameless navigation or with a stereotactic system during harvesting a biopsy.

In prior art are the harvested tissue samples immediately sent to the pathological department for analysis, and the results must be received before the surgical procedure can be closed. In case the biopsies do not show diagnostic results, the surgery needs to be proceeded for an additional tissue harvesting along a second trajectory. The waiting time is usually about one hour, and this time and a second or third or fourth trajectory can be avoided by using the probe-guide and combinable devices presented here. Taking a biopsy which gives the results immediately and guides the surgeon in harvesting the biopsies from the most likely tumor locations. The harvested tissue is thereafter sent for more accurate postoperative analysis. In summary, the advantages with the present system provides a device and method that identifies malign tissue direct without having to send the biopsy for preliminary examination.

1. A probe-guide (1) for guiding, identifying, and harvesting a biopsy, the probe-guide (1) is substantially tube-shaped, having a distal and a proximal end wherein the distal end is for insertion into a body and the proximal end is for connection to an energy source, wherein the wall of the probe-guide (1) comprises a first (e) and a second (f) opening, the first opening (e) is arranged in the distal end or tip,
the second opening (f) is arranged at a distance from the distal end or tip, preferably arranged in the sidewall of the probe-guide (1), and wherein
the probe-guide (1) is designed to receive another device into its lumen such as:
iii) a removable optical probe (2) for optical guidance and/or identification of malign tissue, wherein the probe (2) is essentially tube-shaped outer housing accommodating optical fibers, the probe (2) has a distal and a proximal end wherein the distal end is for insertion into a body and the proximal end is for connection to an energy source, optical fiber pairs are arranged in close proximity towards the distal end, the optical fibers at least 4 and the diameter of the fibers are in the range of 125 to 250 μm, the probe (2) is removably arranged into the lumen of the probe-guide (1) configured to transmit and receive light via the first opening (e), and/or
iv) a removable biopsy needle (3) for harvesting a biopsy, wherein the biopsy needle (3) is removably arranged into the lumen of the probe-guide (1) configured to harvest a biopsy via the second opening (f),
wherein the probe (2) and the biopsy needle (3) are not arranged in the lumen of the probe-guide (1) simultaneously.

2. The probe-guide (1) according to claim 1, wherein the length a of the probe-guide (1) is in the range of 100-300 mm, and the width (diameter) (b) is in the range of 1.5-3 mm.

3. The probe-guide (1) comprising the probe (2) according to claim 1, wherein the length (c) of the probe (2) is in the range of 50-400 mm.

4. The probe-guide (1) comprising the probe (2) according to claim 1, wherein the probe (2) comprises a light spectrometer laser (10); and
a LDF laser (12); and
a Raman laser (14); and
a spectrometer detector (11); and
a laser doppler detector (13); and
a Raman detector (15).

5. The probe-guide (1) and probe (2) according to claim 1, wherein the material of the probe-guide (1), and probe (2) is medical steel.

6. The probe-guide (1) according to claim 1, wherein a pointer for electromagnetic navigation is arranged into the lumen of the probe-guide (1).

7. The probe-guide (1) according to claim 1, wherein a device (4) for delivering a medicament is arranged into the lumen of the probe-guide (1).

8. A method for guiding, identifying, and harvesting a biopsy comprising the following steps:
xiv) mounting a stereotactic device or frameless navigation system;
xv) drilling a hole at a predetermined site in a skull (predicted from the MR-images);
xvi) moving the probe-guide (1) comprising a removable optical probe (2) according to claim 1 to the pre-drilled hole via a navigation system or by an electrical or hand driven mechanical device, or by hand, or by a robot navigation system;
xvii) deploying the probe-guide (1) comprising the probe (2) into the pre-drilled hole with or without a supporting device;
xviii) measuring and recording the microvascular perfusion, TLI and fluorescence stepwise with or without an electrical or mechanical insertion device or continuously along the way to the target;
xix) pushing the probe probe-guide (1) forward and record if the microcirculation is within a valid level meaning that no vessels are detected in front of the probe-guide (1);
xx) determining where the biopsy is to be taken by measuring and recording emitted fluorescence light (every mm) before and throughout the tumour trajectory wherein the emitted fluorescence signal is illustrated as a peak or as an audio signal, where the high fluorescence signal indicates highly malignant tissue of said tumour, and
xxi) removing the probe (2) from the probe-guide (1),
xxii) arranging the biopsy-needle (3) into the lumen of the probe-guide (1),
xxiii) harvest the biopsy via the opening (f) in the sidewall of the probe-guide (1),
xxiv) removing the probe-guide (1) comprising the biopsy needle (3)
xxv) sewing the wound together, and
sending the biopsy for examination.

9. The method for guiding, identifying, and harvesting a biopsy comprising steps i)-ix) according to claim 8 and comprising the steps of:
removing the biopsy needle (4) in step xi);
arranging a device (4) comprising a medicament in the lumen of the probe-guide (1);
delivering the medicament to the target site, and
sewing the wound together.

10. A real time system for localizing, identifying, and harvesting a tumor biopsy, said system comprising:
v) a probe-guide (1), a removably probe (2), a removably biopsy-needle (3) according to claim 1,
vi) a stereotactic or frameless system,
vii) a control device, and
viii) an analysis system.

11. The probe-guide (1) according to claim 1, comprising openings (e), (f), for transmitting signals from a device arranged or inserted into the lumen of the probe-guide (1), for removal of tissue by vaporization, abrasion, or destruction.
